(19) United States
(12) Patent Application Publication
(10) Pub. No.: US 2020/0188256 A1
(43) Pub. Date: Jun. 18, 2020

(54) TRANSPARENT HAIR CONDITIONING COMPOSITION COMPRISING BIS-(ISOSTEAROYL/OLEOYL ISOPROPYL) DIMONIUM METHOSULFATE

(71) Applicant: Henkel AG & Co. KGaA, Duesseldorf (DE)

(72) Inventors: Marlene Battermann, Asendorf (DE); Thomas Schroeder, Hamburg (DE)

(73) Assignee: Henkel AG & Co. KGaA, Duesseldorf (DE)

(21) Appl. No.: 16/688,289

(22) Filed: Nov. 19, 2019

(30) Foreign Application Priority Data
Dec. 17, 2018 (DE) 10 2018 221 974.5

(51) Int. Cl. A61K 8/37; A61Q 5/12; A61K 8/42; A61K 8/34

(52) U.S. Cl. CPC A61K 8/37; A61K 8/345; A61K 8/42; A61Q 5/12

(57) ABSTRACT

A silicone-free, transparent hair conditioning composition comprising bis-(isostearoyl/oleoyl isopropyl) dimonium methosulfate, a cosmetic product incorporating the composition, and methods of using the composition.

### CROSS-REFERENCE TO RELATED APPLICATION

This application claims priority to German Patent Application No. 10 2018 221 974.5, filed Dec. 17, 2018, which is incorporated herein by reference in its entirety.

### TECHNICAL FIELD

The present disclosure generally relates to hair care compositions, and more particularly relates to a silicone-free, transparent hair conditioning composition comprising bis-(isostearoyl/oleoyl isopropyl) dimonium methosulfate. The present disclosure also relates to the use of the compositions for the care of keratinous fibers.

### BACKGROUND

Hair care compositions are used to style, cleanse and provide a pleasant feel to the hair. Such compositions come in a variety of forms, and include shampoos, conditioners, leave-on conditioning sprays and styling products.

Some hair care compositions, such as conditioners, are used to moisturize and condition the hair, to restore some of the moisture that was removed by a cleansing or shampoo composition. Such compositions may include conditioning agents, which act to retain moisture in the hair by a variety of mechanisms. Some conditioning agents, such as dimethicone, form a barrier and act to prevent the evaporation of moisture from the hair. Other agents, such as propylene glycol, act as humectants to attract moisture to the hair. In each case, the benefit persists only as long as the benefit agent is present on the hair. As such, providing an encapsulated benefit agent may provide increased benefits to hair feel and appearance by providing an extended release of the benefit agent by continuing to deposit the benefit agent onto the hair as the hair is touched, combed, brushed, or otherwise manipulated.

Additional hair care compositions are leave-in compositions. These include styling gels, hairsprays, creams, pomades, or waxes. Such compositions may also include benefit agents, such as moisturizing ingredients, conditioning ingredients, and illuminating ingredients. These benefit agents act immediately upon contact with the hair but may decrease in efficacy over time. As a result, providing an encapsulated benefit agent may assist the benefit agent to provide increased benefit to hair feel and appearance by providing a gradual deposit of the benefit agent onto the hair as the hair is physically manipulated and the capsules are opened, releasing the benefit agent onto the hair.

In addition to the natural environmental influences, human hair is exposed to a number of other, in particular cosmetic, stresses. To these, the strains include, for example, the coloring of the hair and its deformation, for example by a perm. To reduce the adverse effects of the hair structure affecting (environmental) influences, but also to maintain and improve the natural hair structure cosmetic hair products are used. An essential active ingredient in many of these cosmetics are the organosilicon compounds, in particular the silicones such as trisiloxanes, which are exemplified by nourishing properties. The disadvantages of these silicones are the reduced penetration of active ingredients and auxiliaries into the hair due to wetting of the hair surface and the hair styling being hampered by the wetting of the hair surface. Further, due to their persistence in the environment, it is preferable to dispense with the use of cyclomethicones.

The provision of low-silicone or silicone-free care products is therefore a relevant task in the field of hair cosmetics. However, silicone-free leave-on products do not always exhibit the same caring properties as silicone-based products, and it is a challenge to formulate cosmetically acceptable formulations at the care levels of silicones, without using silicones.

Accordingly, it is desirable to provide a hair conditioning composition that is silicone-free and that has enhanced care properties.

### BRIEF SUMMARY

The inventors have surprisingly found that a transparent hair composition can be formulated, which is advantageously silicone-free. The composition comprises bis-(isostearoyl/oleoyl isopropyl) dimonium methosulfate and optionally an alcohol.

The silicone-free, transparent composition comprising bis-(isostearoyl/oleoyl isopropyl) dimonium methosulfate was particularly shown to achieve the beneficial care levels, especially compared to a commercially available leave-on silicone oil-based formulation.

Further details as to options for the components are disclosed herein.

Compositions according to the present specification are suitably provided in the form of a spray. The spray may also contain additional components, such as fragrances to provide the user with a sensory awareness that the benefit agent has been released.

There is also provided a method of conditioning keratin fibers, the method comprising contacting a keratin fiber with a conditioning-effective amount of a composition as disclosed herein.

There is also provided a method of conditioning keratin fibers, comprising contacting a plurality of keratin fibers with an amount of the composition as disclosed herein effective to increase the volume of the keratin fibers on styling.

There is also provided a method for conditioning keratin fibers, wherein the hair composition as disclosed herein is spray-applied onto wet or dry keratin fibers and left on the keratin fibers until they are next washed.

There is also provided a cosmetic preparation comprising the composition as disclosed herein in a transparent package suitable for dispensing the agent in the form of uniform, small droplets.

### DETAILED DESCRIPTION

The following detailed description of the present disclosure is merely exemplary in nature and is not intended to limit the present disclosure or the application and uses of the present disclosure. Furthermore, there is no intention to be bound by any theory presented in the preceding background or the following detailed description.

As used herein, the term “about” refers to amounts, concentrations or values that are within about 10 percent of the expressed amount, concentration or value. Additionally, all provided concentration ranges are intended to include all possible concentration ranges contained within the provided range.

As noted above, there is provided a transparent hair conditioning composition comprising bis-(isostearoyl/oleoyl isopropyl) dimonium methosulfate, wherein the composition is silicone-free. In the context of the present disclosure, “silicone-free” is taken to mean that the composition is free from organosilicon compounds, in particular free from silicones, trisiloxanes, and silicone oils.

The transparent hair conditioning composition suitably comprises bis-(isostearoyl/oleoyl isopropyl) dimonium methosulfate in an amount ranging from about 0.01 to about 5.0% by weight of the total composition. Preferably, the bis-(isostearoyl/oleoyl isopropyl) dimonium methosulfate is present in an amount ranging from about 0.1 to about 3.0% by weight of the total composition, preferably from about 0.2 to about 2.0% by weight of the total composition, more preferably from about 0.5 to about 2.0% by weight of the total composition.

The transparent hair conditioning composition may further comprise a dicarboxylic acid, or a salt or derivative thereof or a mixture of different dicarboxylic acids/salts/derivatives thereof. The dicarboxylic component may be selected from any suitable dicarboxylic component that exhibits a care effect. Suitable ingredients falling within this component include dicarboxylic acids having 2 to 10 carbon atoms. The or each dicarboxylic acid may be selected from oxalic acid, malonic acid, succinic acid, glutaric acid, adipic acid, pimelic acid, azelaic acid, maleic acid, fumaric acid, sorbic acid and mixtures thereof. The preferred component (ii) is succinic acid together with one or more different dicarboxylic acids/salts/derivatives thereof. More preferably, the dicarboxylic acid is succinic acid.

The transparent hair conditioning composition may further comprise a cationic surfactant or a mixture of different cationic surfactants. The cationic surfactants are selected from amidoamines, permanent cationic amidoamines, and mixtures thereof. Thus, the cationic surfactant component may be one amidoamine or a mixture of different amidoamines. The cationic surfactant component may alternatively be one permanent cationic amidoamine or a mixture of different permanent cationic amidoamines. The cationic surfactant component may alternatively be one or more amidoamines in combination with one or more permanent cationic amidoamines.

When an amidoamine is present, it may be selected from one or more of Brassicamidopropyl dimethylamine, lauramidopropyl dimethylamine, myristamidopropyl dimethylamine, stearamidopropyl dimethylamine, cocamidopropyl dimethylamine, ricinolamidopropyl dimethylamine, isostearamidopropyl dimethylamine, oleamidopropyl dimethylamine, behenamidopropyl dimethylamine and palmamidopropyl dimethylamine, preferably Brassicamidopropyl dimethylamine.

When a permanent cationic amidoamine is present, it may be selected from one or more of bis-(Isostearoyl/Oleoyl Isopropyl) dimonium methosulfate, Cetrimonium Methosulfate, Distearoylethyl Hydroxyethylmonium Methosulfate, Quaternium-33, behenamidopropyl ethyldimonium ethosulfate, behenamidopropyl PG-dimonium chloride, oleamidopropyl ethyldimonium ethosulfate, oleamidopropyl PG-dimonium chloride, cocamidopropyl ethyldimonium ethosulfate, cocamidopropyltrimonium chloride, ricinoleamidopropylethyldimonium ethosulfate, rinoleamidopropyltrimonium chloride, ricinoleamidopropyltrimonium methosulfate, stearamidopropyl ethyldimonium ethosulfate, stearamidopropyl trimonium methosulfate, undecyleneamidopropyltrimonium methosulfate, lauramidopropyl PG-dimonium chloride, canolamidopropyl ethyldimonium ethosulfate, preferably Bis-(Isostearoyl/Oleoyl Isopropyl) Dimonium Methosulfate and/or Cetrimonium Methosulfate and/or Distearoylethyl Hydroxyethylmonium Methosulfate.

Suitably, the cationic surfactant component comprises brassicamidopropyl dimethylamine. A suitable combination of ingredients falling within component (iii) is brassicamidopropyl dimethylamine, distearoylethyl hydroxyethylmonium methosulfate, and cetrimonium methosulfate. Component (iii) may also be a combination of brassicamidopropyl dimethylamine, bis-(isostearoyl/oleoyl isopropyl) dimonium methosulfate, distearoylethyl hydroxyethylmonium methosulfate, and cetrimonium methosulfate.

The composition disclosed herein may contain a total amount of cationic surfactants ranging from about 0.01% to about 10%, such as from about 0.1% to about 8%, relative to the total weight of the composition. Preferably, the total amount of cationic surfactants ranges from about 0.1% to about 2%, more preferably from about 0.5% to about 1.5%, relative to the total weight of the composition.

The transparent hair conditioning composition may further comprise an aqueous-alcoholic carrier. The alcoholic component of the aqueous-alcoholic carrier may be selected from C1-C6 linear or branched chain alcohols, C1-C6 linear or branched chain diols, polyhydric alcohols and mixtures thereof. Preferably, the alcoholic component of the aqueous-alcoholic carrier is selected from alcohol, alcohol denat, propyl alcohol, isopropyl alcohol, ethylene glycol, propylene glycol, butylene glycol, pentylene glycol, hexylene glycol, glycerin, sorbitol, and mixtures thereof. More preferably, the carrier is a mixture of water, alcohol denat, propylene glycol and glycerin.

The composition disclosed herein may contain a total amount of alcohol ranging from about 5% to about 25%, such as from about 7% to about 20%, relative to the total weight of the composition. Preferably, the total amount of alcohol ranges from about 10% to about 20%, relative to the total weight of the composition.

The hair conditioning composition may further comprise one or more glyceryl esters or derivatives thereof (such ingredient excluding fats and oils). The glyceryl ester/derivative component may be an ester of a polyglycerin with an acid or a mixture of acids. The ester may be a monoester, diester, triester, tetra ester, pentaester and so on. The acid may be selected from behenic acid, capric acid, caprylic acid, olive acid, succinic acid, coconut acid, linoleic acid, dilinoleic acid, myristic acid, stearic acid, isostearic acid, hydroxystearic acid, lauric acid, oleic acid, palmitic acid, erucic acid, palm acid, palmitic acid, ricinoleic acid, decanoic acid and Undecylenic Acid (and mixtures thereof). The polyglycerin may be selected from Polyglycerin-3, Polyglycerin-4, Polyglycerin-5, Polyglycerin-6, Polyglycerin-8, Polyglycerin-10, Polyglycerin-15 and Polyglycerin-20.

Suitable glyceryl ester/derivative components include Polyglyceryl-3 Caprate/Caprylate/Succinate, Polyglyceryl-6 Caprylate, Polyglyceryl-3 Cocoate, Polyglyceryl-6 Ricinoleate, and Polyglyceryl-4 Caprate. Preferably, the composition comprises two or more such glyceryl esters or derivatives. More preferably, the composition comprises three or four such glyceryl esters or derivatives. In one example, the composition comprises Polyglyceryl-3 Caprate/Caprylate/Succinate, Polyglyceryl-6 Caprylate, Polyglyceryl-3 Cocoate, Polyglyceryl-6 Ricinoleate, and Polyglyceryl-4 Caprate.

The hair conditioning composition may comprise bis-(isostearoyl/oleoyl isopropyl) dimonium methosulfate, brassicamidopropyl dimethylamine and succinic acid. The carrier of such a composition may be a water-alcohol (suitably alcohol denat.) mix, including glycerin and propylene glycol. The composition may further comprise a mixture of glyceryl esters/derivatives (excluding fats and oils), such as a mixture of two or more of Polyglyceryl-Caprate/Caprylate/Succinate, Polyglyceryl-6 Caprylate, Polyglyceryl-3 Cocoate, Polyglyceryl-6 Ricinoleate and Polyglyceryl-4 Caprate. The composition may also comprise a natural oil, such as Persea Gratissima Oil.

The hair conditioning composition suitably further comprises one or more further surfactants, i.e. a cationic, nonionic, amphoteric, or zwitterionic surfactant (any additional cationic surfactant being different to that required by component (iii)). Anionic surfactants may have a negatively charged hydrophilic end. Examples of anionic surfactants include sulfate, sulfonate, carboxylate, phosphate, or the like. Anionic surfactants may be sensitive to water hardness. Cationic surfactants may be those that have a positively charged hydrophilic end, such as a quaternary amine. Nonionic surfactants may have a hydrophilic end which may be charge neutral, such as an ethoxylate, glycoside, or polyol; such surfactants may not be sensitive to water hardness. Amphoteric surfactants may be those that have a hydrophilic end which has a functional group that is capable of acting as a base, and a functional group that is capable of acting as an acid, such as amine oxides. Zwitterionic surfactants may have both a positive and negative charge on their hydrophilic ends, such as sultaines, or betaines. The hydrophobic end may include a saturated or unsaturated, linear or branched, substituted or unsubstituted, cyclic or acyclic, alkyl or silyl chain containing at least 8 carbon or silicon atoms.

The hair conditioning composition as contemplated herein preferably further comprises one or more nonionic surfactants. The or each nonionic surfactant suitably comprises or includes an ester of a fatty acid. The or each nonionic surfactant may comprise or include Glycol Distearate.

The composition disclosed herein may contain a total amount of nonionic surfactant ranging from about 0.1% to about 5%, such as from about 0.5% to about 3%, relative to the total weight of the composition. Preferably, the total amount of nonionic surfactant ranges from about 0.5% to about 2.5%, relative to the total weight of the composition.

The hair conditioning composition as contemplated herein suitably further comprises one or more solubilizers. The or each solubilizer may be an ethoxylation product of optionally hardened vegetable and animal oils. The or each solubilizer may be ethoxylated mono-, di- and triglycerides of C8-22 fatty acids with from about 4 to about 50 ethylene oxide units, for example hydrogenated ethoxylated castor oil, olive oil ethoxylate, almond oil ethoxylate, mink oil ethoxylate, polyoxyethylene glycol, caprylic/capric acid glycerides, polyoxyethylene glycerol monolaurate and polyoxyethylene glycol coconut fatty acid glycerides. Preferably, the solubilizer comprises or includes Caprylic/Capric Triglyceride.

The composition disclosed herein may contain a total amount of solubilizers ranging from about 0.1% to about 5%, such as from about 0.5% to about 3%, relative to the total weight of the composition. Preferably, the total amount of solubilizers ranges from about 0.5% to about 2.5%, relative to the total weight of the composition.

The hair conditioning composition according to the present disclosure may further comprise one or more further benefit agents. Suitable benefit agents are selected from moisturizing ingredients, conditioning ingredients, illuminating ingredients antidandruff agents, preservatives, UV filters, fats and oils, thickeners, polymers, humectants, vitamins and/or provitamins, hair structuring agents, hair care agents, hair restorers and agents for combating hair loss.

A moisturizing ingredient may be any component that is capable of moisturizing the hair, and include occlusive-type moisturizers, oils, esters, humectants, and vitamins that may strengthen the hair to benefit the hair's natural moisture retention abilities.

A conditioning ingredient may be any component that is capable of improving the moisture or feel of the hair, and include the aforementioned moisturizing ingredients, proteins, and hydrolyzed proteins.

Illuminating ingredients may be any component that provides a sparkling visual effect, and include mica, pearlescent pigments, and glitter particles.

Oils are nonpolar substances that are viscous liquids at room temperature (from about 20-25° Celsius). Non-limiting examples of suitable oils include natural oils such as triglycerides and petroleum-derived hydrocarbons, synthetic oils such as hydrocarbons, and semi-synthetic oils such as hydrogenated or chemically modified triglycerides.

Non-limiting examples of specific oils which may be used in a composition according to the present disclosure include Persea Gratissima Oil, coconut oil, Prunus Armeniaca Kernel Oil, palm oil, castor oil, meadowfoam seed oil, vegetable oil, animal fats such as tallow, hydrogenated analogs of the foregoing, paraffinum liquidum, and combinations and mixtures thereof.

The composition disclosed herein may contain a total amount of oils ranging from about 0.01% to about 5%, such as from about 0.01% to about 3%, relative to the total weight of the composition. Preferably, the total amount of oils ranges from about 0.1% to about 2%, relative to the total weight of the composition.

Any ester-based moisturizing ingredient may be suitable for inclusion in a composition according to the present disclosure. There may be some overlap between ester-based moisturizing ingredients and oil-based moisturizing ingredients. Non-limiting examples of suitable ester-based moisturizing ingredients include monoesters and polyesters, including triglyceride oils. Non-limiting examples of specific oils which may be used in a composition according to the present disclosure include acetylated glycol stearate, arachidyl behenate, butyl avocadate, butyl myristate, cetearyl palmate, coco-caprylate, decyl oleate, dipentaerythrityl hexahydroxystearate, ethyl oleate, isocetyl myristate, propylene glycol stearate, diisopropyl adipate, polysorbates, and combinations and mixtures thereof.

Any humectant may be suitable for inclusion in a composition according to the present disclosure. Humectants are hygroscopic substances that attract ambient moisture. Non-limiting examples of suitable humectants include glycerol (INCI name glycerine), acetyl arginine, betaine, lauryl malamide, mannitol, propylene glycol, xylose, polymeric humectants such as lauryl methyl gluceth-10 hydroxypropyldimonium chloride, and combinations and mixtures thereof. Preferably, the composition disclosed herein further comprises propylene glycol and/or glycerol, more preferably both propylene glycol and glycerol.

Any vitamins that strengthen the hair, whereby benefiting the hair's natural moisture retention abilities may be suitable for inclusion in a composition according to the present disclosure. Other vitamins that may be suitable are those that modulate hair rejuvenation or growth. For example, suitable vitamins include retinol (vitamin A), thiamine (vitamin B1), riboflavin (vitamin B2), niacin (vitamin B3), pantothenic acid (vitamin B5), pyridoxine (vitamin B6), biotin (vitamin B7), folic acid (vitamin B9), cyanocobalamin (vitamin B12), ascorbic acid (vitamin C), tocopherol (vitamin E), and combinations and derivatives thereof.

Any proteins that provide a conditioning effect to the hair, whereby benefiting the hair's appearance or feel may be suitable for inclusion in a composition according to the present disclosure. Proteins are natural polymers produced by cellular machinery; proteins are amino acid chains held together through amide bonds, and may include crosslinks, secondary, and/or tertiary structure as a result of the amino acids present, and the order of the amino acids in the protein. Proteins may also be modified, such as by attachment of sugars (glycosylation); such modification may occur enzymatically or chemically. Non-limiting examples of proteins include keratin, silk, wheat protein, glycoprotein, lactabumin, soy protein, collagen, and combinations thereof.

Any hydrolyzed protein that provides a conditioning effect to hair may be suitable for inclusion in a composition according to the present disclosure. Hydrolyzed proteins are protein chains that have been cleaved along the amide backbone; hydrolysis may be enzymatic, acid-catalyzed, or base-catalyzed. Non-limiting examples of suitable hydrolyzed proteins include hydrolysis products of the above proteins.

Any mica that is capable of providing an optical effect may be suitable for inclusion in a composition according to the present disclosure. Micas are a class of phyllosilicate minerals. Non-limiting examples of micas include muscovite, biotite, leipdolite, phlogopite, zinnwaldite, clintonite, illite, phengite, and combinations thereof.

Any pearlescent pigment that is capable of providing an optical effect on the hair may be suitable for inclusion in a composition according to the present disclosure. Pearlescent pigments are particulate pigments that have a shiny or glossy appearance. In some examples, pearlescent pigments provide different colors based on the angle of incident light and the angle of observation. Non-limiting examples of pearlescent pigments include mica coated with metal oxides, aluminum oxide coated with metal oxides, bismuth oxychloride, and combinations thereof.

Any glitter particles that are capable of providing an optical effect on the hair may be suitable for inclusion in a composition according to the present disclosure. Glitter may be flat particles that reflect light at different angles, causing the particles to shimmer. Non-limiting examples of glitter particles include plastic-based glitters, glitter derived from stones such as malachite, glitter derived from natural sources such as insect-based glitters and glass-based glitters.

The hair conditioning composition according to the present disclosure may also include additional components. Such additional components may include a suspending polymer, a wax, a preservative, a fragrance, a deposition aid, and combinations thereof. The composition may also contain additives known to an individual skilled in the art, such as a dye, a pigment, an antibacterial agent, a foaming agent, a plant extract, plant matter, a chelator, an alkali metal halide, and combinations thereof.

A wax is a hydrophobic solid that is malleable at or near room temperature (about 20-25° Celsius). A wax may be included in a composition according to the present disclosure as a carrier, viscosity modifier, hair styling agent, or adjuvant. A wax may be capable of shaping the hair, as well as acting as a moisturizing ingredient or conditioner due to its hydrophobic nature. Non-limiting examples of types of waxes include esters of fatty alcohols with fatty acids, sterol esters, and hydrocarbons. Non-limiting examples of waxes include beeswax, lanolin, sunflower seed wax, carnauba wax, montan wax, candelilla wax, paraffin wax, tallow tree wax, ceresin wax, and derivatives and combinations thereof.

Preservatives are compounds that may be added to the composition disclosed herein to prevent undesirable decomposition over time. Non-limiting examples of preservatives include benzoic acid, benzyl alcohol, phenol, phenoxyethanol, formaldehyde, glyoxal, DMDM hydantoin, cresol, para-hydroxybenzoic acid and esters thereof, chlorhexidine, propionic acid, and salts and/or combinations thereof. The composition disclosed herein may contain up to about 5% by weight of preservatives, relative to the total weight of the composition. For example, the composition disclosed herein may contain from about 0.01% to about 3%, such as from about 0.05% to about 2% of preservatives, relative to the total weight of the composition.

A fragrance comprises a number of ingredients; fragrance ingredients are compounds that provide the composition with a pleasing scent. While fragrances may often be complex mixtures of fragrance ingredients, it is also possible to include a single fragrance ingredient in a composition. The olfactory intensity of a fragrance ingredient may be a function of the concentration of the fragrance ingredient, the vapor pressure of the fragrance ingredient, and the potency of the interactions between the fragrance ingredient and a user's olfactory receptors. A fragrance may be incorporated into the composition disclosed herein along with the benefit agent in order to provide a user with a sensory awareness that the moisturizing ingredient has been deployed onto the hair. Alternatively, a fragrance may also be incorporated into the composition disclosed herein directly. Fragrance ingredients are generally known by an individual skilled in the art, and include ketones, aldehydes, esters, and the like. Fragrance ingredients also include naturally-occurring plant and animal oils. Non-limiting examples of suitable fragrance ingredients include linalool, hexyl salicylate, citronellol, butylphenyl methylpropional, limonene, allylanisole, carvone, nonalactone, liffarome, 2,4-dimethyl-3-cyclohexene carboxaldehyde, adoxal, galaxolide, methyl benzoate, geraniol, camphor, citral, eucalyptol, alpha-damascone, florhydral, and the like. Derivatives and combinations thereof may also be suitable for inclusion.

The composition according to the present specification may contain a fragrance in an amount of up to about 10% by weight, such as from about 0.001% to about 5% by weight, relative to the weight of the total composition. For example, the composition according to the present specification may contain a fragrance in an amount of from about 0.01% to about 4% by weight, or from about 0.05% to about 2% by weight, relative to the total weight of the composition.

Dyes and pigments are compounds which confer color to a composition or to the hair to which a dye or pigment is applied. Dyes and pigments may be added to the composition disclosed herein in order to imbue the composition with a consumer-acceptable color. In another example, a dye or pigment is added to the composition disclosed herein in order to provide a simultaneous coloring effect to the hair. Non-limiting examples of dyes and pigments which may be used to color the composition disclosed herein include titanium dioxide, mica, iron oxides, violet 2, red 4, red 6, red 7, red 33, red 40, blue 1, blue 4, yellow 5, yellow 6, yellow 10, orange 4, orange 5, orange 10, vat red 1, vat blue 1, vat blue 4, vat blue 6, vat orange 7, vat violet 2, and combinations thereof.

Dyes and pigments may also include permanent dyes, and direct dyes, which may be either semi-permanent dyes or temporary dyes. Permanent dyes may include oxidative dye precursors, which react with oxidizing agents, such as hydrogen peroxide, to form dye molecules on the hair. Oxidative dye precursors include oxidation bases, which include para- and ortho-substituted aromatic rings; these may be supplemented by couplers, which modify the color produced by the oxidation dye precursors and include meta-substituted aromatic rings. Semi-permanent dyes include anthraquinones, other multicyclic ring systems, as well as monocyclic compounds that confer color directly, such as 4-amino-3-nitrophenol. Temporary hair dyes confer color to the hair only until the hair is washed, and include inorganic pigments comprising titanium dioxide, mica, iron oxides, and/or ultramarines, as well as cationic dyes such as basic blue 99 and basic brown 17. Dyes and pigments may be used in any suitable quantity, including up to about 20% by weight, relative to the total weight of the composition. For example, the composition disclosed herein may contain from about 1% to about 20% by weight, such as from about 4% to about 15% by weight of dyes and/or pigments, relative to the total weight of the composition. In one example in which the composition contains a number of permanent dyes, the composition may also be mixed with another composition containing from about 2% to about 30% by weight, such as from about 6% to about 20% of an oxidant, such as hydrogen peroxide.

A composition disclosed herein may also include at least one antibacterial agent. An antibacterial agent is any agent that assists in the removal of bacteria, kills bacteria, or arrests bacterial growth. Some antibacterial agents also have additional functions and belong to one or more than one of the aforementioned or following classes. Suitable non-limiting examples of antibacterial agents include antiseptics, triclosan, triclocarban, usnic acid salts, benzethonium salts, benzalkonium salts, compounds which inhibit the 70S (bacterial) ribosome, and compounds which reduce the integrity of the bacterial cell wall. Additional non-limiting examples of antibacterial agents include ethanol, isopropanol, aminoglycosides (such as neomycin), cephalosporins (such as cefalexin), lincosamides (such as lincomycin), tetracyclines (such as doxycycline), penicillins (such as amoxicillin), and combinations thereof. A composition as disclosed herein may contain an antibacterial agent in any appropriate amount, such as from about 0.1% to about 75% by weight. Antibacterial agents such as ethanol and isopropanol may require higher concentrations, such as from about 50% to about 75%, and may also provide the carrier for the composition disclosed herein. In contrast, antibacterial active ingredients such as benzethonium and benzalkonium salts may be effective at lower concentrations, such as from about 0.1% to about 10% by weight, or from about 1% to about 6% by weight, relative to the total weight of the composition.

Foaming agents are compounds that stabilize foams. Foaming agents increase the propensity of a composition to form a foam, and/or may stabilize a foam by inhibiting the coalescence of bubbles within the foam. Certain types of surfactants are capable of acting as foaming agents in the composition disclosed herein; however, not every type of surfactant enhances foam stability. Non-limiting examples of foaming agents include sodium laureth sulfate, cocamidopropyl betaine, cocamidopropyl hydroxysultaine, behenyl betaine, hydroxystearamide MEA, lauramide MEA, myristamide MEA, myristamide DEA, PEG-3 lauramide, PEG-2 lauramine, lauramine oxide, PEG-3 lauramine oxide, and cocamine oxide. A number of foaming agents may be included the composition disclosed herein at concentrations up to about 10% by weight, such as from about 0.1% to about 7% by weight, or from about 0.5% to about 5% by weight, relative to the total weight of the composition.

Plant extracts are natural compounds or mixtures of compounds produced in a plant that contain at least one agent that has either a real or perceived benefit to the skin, or to the composition as a whole. The inclusion of some plant extracts may improve consumer acceptance of a composition on the basis of these benefits, or a consumer preference for naturally produced compositions over synthetically produced compositions. Plant extracts include oils, fragrance ingredients, fatty acids, and/or various other components depending on the extraction methods employed and any subsequent processing that is performed. Non-limiting examples of plant extracts include Prunus Amygdalus dulcis extract, Oenothera biennis extract, Zingiber officinale extract, Jasminum extracts, Lavandula angustifolia extract, Menthapiperita extract, Rosa extracts, Hypericum perforatum extract, and combinations thereof. The composition disclosed herein may contain up to about 20% by weight, such as from about 0.1% to about 15% by weight, or from about 0.5% to about 10% by weight of plant extracts, relative to the total weight of the composition.

Plant matter is plant material which may be incorporated into an example of compositions (202) according to the present specification. Such plant material may provide abrasive properties as exfoliants, fragrance properties, or may be able to act as a thickener. The incorporation of plant matter into a composition may improve consumer acceptance, which may be based on the perception of the natural qualities of the compositions including plant matter. Non-limiting examples of plant matter which may be incorporated in compositions according to the present specification include whole flowers, flower petals, stems, seeds, roots, and fruits.

Chelators are compounds that coordinate metal ions. Chelators may be included in an example of a composition as antibacterial agents, preservatives, pH regulators, or to provide other such properties to the composition. Non-limiting examples of chelators include natural polyacids (such as citric acid), phosphate salts (such as disodium pyrophosphate), bisphosphonates (such as etidronic acid), aminocarboxylic acids (such as ethylenediaminetetraacetic acid (EDTA) and ethylenediamine-N,N′-disuccinic acid (EDDS)), and combinations and/or salts thereof. Chelators may be included in the composition disclosed herein in any suitable amount, such as up to about 5% by weight, relative to the total weight of the composition.

Alkali metal halides are salts of alkali metals and halogen atoms. Alkali metal halides may be included in an example of a composition as thickeners, ionic strength modulators, or to confer other such properties to the composition. Alkali metal halides are neutral compounds. Non-limiting examples of alkali metal halides include lithium chloride, lithium bromide, sodium chloride, sodium bromide, potassium chloride, potassium bromide, and combinations thereof. The composition disclosed herein may contain an alkali metal halide in any suitable amount, such as up to about 5% by weight, relative to the total weight of the composition.

The composition disclosed herein may also contain other, additional components known to an individual skilled in the art. For example, the composition disclosed herein may contain additional polymers, which may be natural, synthetic, and/or semi-synthetic. Natural polymers include hydrolyzed keratin, silk, collagen, honey, cellulose, and the like. Synthetic polymers include acrylates/acrylamide copolymers, acrylamidopropyltrimonium chloride/acrylates copolymers, polyethylene glycol polymers, polypropylene glycol polymers, polyquaterniums, and the like. Semi-synthetic polymers include chemically modified natural polymers, polymers assembled from both natural and synthetic monomers, and the like. In another example, the composition disclosed herein includes an exfoliant, which are particles that do not dissolve in the carrier and act as an abrasive to assist in efficient cleansing of the hair. In a further example, the composition disclosed herein includes a pH buffer, to maintain the pH within a desired range. In another example, the composition disclosed herein includes a pH adjusting agent, which may be provided either with or without a pH buffer. In yet another example, the composition disclosed herein includes an enzyme. In a further example, the composition disclosed herein may contain a UV stabilizer or protectant which may reduce damage to the hair resulting from exposure to the sun. In another example, the composition disclosed herein may contain a bleaching agent.

The composition disclosed herein may have any viscosity that renders it suitable for application to the hair. Suitably, the product is a spray, and the viscosity is suitable for application of the composition in liquid form as a spray. The viscosity of the composition disclosed herein may be measured in centipoise (cps). The composition disclosed herein may have a viscosity in the range of about 0.8 cps to about 500,000 cps. In one example, a composition as disclosed herein is not subjected to a foaming operation upon dispensing and is offered as a sprayable liquid with a viscosity in the range of about 1,000 to about 5,000 cps, such as from about 2,000 cps to about 4,000 cps.

### Methods

The present specification is also directed to a method for using a composition. The method includes applying the hair conditioning composition as disclosed herein to the hair. The method includes spraying the hair conditioning composition as disclosed herein onto the hair. The method may additionally include touching or rubbing the hair.

The method for using a hair conditioning composition as disclosed herein may also include additional, optional steps. In one example, such steps may include air drying the hair, drying the hair using heat, styling the hair, and any other suitable step that is known to an individual skilled in the art of hair composition.

The subject matter disclosed herein may be defined by the following numbered statements.

1. A transparent hair conditioning composition comprising bis-(isostearoyl/oleoyl isopropyl) dimonium methosulfate, wherein the composition is silicone-free.
2. A transparent hair conditioning composition according to statement 1, comprising bis-(isostearoyl/oleoyl isopropyl) dimonium methosulfate in an amount ranging from about 0.01 to about 5.0% by weight of the total composition.
3. A transparent hair conditioning composition according to statement 1 or 2, comprising bis-(isostearoyl/oleoyl isopropyl) dimonium methosulfate in an amount ranging from about 0.1 to about 3.0% by weight of the total composition.
4. A transparent hair conditioning composition according to statement 1 or 2, comprising bis-(isostearoyl/oleoyl isopropyl) dimonium methosulfate in an amount ranging from about 0.2 to about 2.0% by weight of the total composition, more preferably from about 0.5 to about 2.0% by weight of the total composition.
5. A transparent hair conditioning composition according to any preceding statement, further comprising at least one dicarboxylic acid, or a salt or derivative thereof.
6. A transparent hair conditioning composition according to statement 5, wherein the or each at least one dicarboxylic acid, or salt or derivative thereof is in the form of a dicarboxylic acid.
7. A transparent hair conditioning composition according to statement 6, wherein the or each dicarboxylic acid has 2 to 10 carbon atoms.
8. A transparent hair conditioning composition according to statement 5 or 6, wherein the or each dicarboxylic acid or salt or derivative thereof is selected from oxalic acid, malonic acid, succinic acid, glutaric acid, adipic acid, pimelic acid, azelaic acid, maleic acid, fumaric acid, sorbic acid and mixtures thereof.
9. A transparent hair conditioning composition according to any one of statements 5 to 8, wherein the dicarboxylic acid or salt or derivative thereof is succinic acid.
10. A transparent hair conditioning composition according to any one of statements 5 to 9, comprising a total amount of dicarboxylic acid or salt or derivative thereof in an amount ranging from about 0.01 to about 5.0% by weight of the total composition.
11. A transparent hair conditioning composition according to any one of statements 5 to 10, comprising a total amount of dicarboxylic acid or salt or derivative thereof in an amount ranging from about 0.1 to about 4.0% by weight of the total composition, more preferably from about 0.3 to about 3.0%, yet more preferably from about 0.5 to about 2.0% by weight of the total composition.
12. A transparent hair conditioning composition according to any preceding statement, further comprising at least one additional cationic surfactant selected from an amidoamine, a permanent cationic amidoamine, and mixtures thereof.
13. A transparent hair conditioning composition according to statement 12, wherein the at least one additional cationic surfactant comprises at least one amidoamine.
14. A transparent hair conditioning composition according to statement 12 or 13, wherein the or each amidoamine is selected from Brassicamidopropyl dimethylamine, lauramidopropyl dimethylamine, myristamidopropyl dimethylamine, stearamidopropyl dimethylamine, cocamidopropyl dimethylamine, ricinolamidopropyl dimethylamine, isostearamidopropyl dimethylamine, oleamidopropyl dimethylamine, behenamidopropyl dimethylamine and palmamidopropyl dimethylamine.
15. A transparent hair conditioning composition according to any one of statements 12 to 14, wherein the amidoamine is Brassicamidopropyl Dimethylamine.
16. A transparent hair conditioning composition according to any one of statements 12 to 15, wherein the at least one additional cationic surfactant comprises at least one permanent cationic amidoamine.
17. A transparent hair conditioning composition according to any one of statements 12 to 16, wherein the or each permanent cationic amidoamine is selected from Cetrimonium Methosulfate, Quaternium-33, behenamidopropyl ethyldimonium ethosulfate, behenamidopropyl PG-dimonium chloride, oleamidopropyl ethyldimonium ethosulfate, oleamidopropyl PG-dimonium chloride, cocamidopropyl ethyldimonium ethosulfate, cocamidopropyltrimonium chloride, ricinoleamidopropylethyldimonium ethosulfate, rinoleamidopropyltrimonium chloride, ricinoleamidopropyltrimonium methosulfate, stearamidopropyl ethyldimonium ethosulfate, stearamidopropyl trimonium methosulfate, undecyleneamidopropyltrimonium methosulfate, lauramidopropyl PG-dimonium chloride, canolamidopropyl ethyldimonium ethosulfate, and mixtures thereof.
18. A transparent hair conditioning composition according to any one of statements 12 to 17, comprising a total amount of the or each additional cationic surfactant ranging from about 0.1 to about 2.0% by weight of the total composition, preferably from about 0.5 to about 1.5%.
19. A transparent hair conditioning composition according to any preceding statement, further comprising an aqueous-alcoholic carrier.
20. A transparent hair conditioning composition according to statement 19, wherein the alcoholic component of the aqueous-alcoholic carrier is selected from C₁-C₆ linear or branched chain alcohols.
21. A transparent hair conditioning composition according to statement 19 or 20, wherein the alcoholic component of the aqueous-alcoholic carrier is selected from alcohol, alcohol denat, propyl alcohol, and isopropyl alcohol.
22. A transparent hair conditioning composition according to any one of statements 19 to 21, wherein the carrier comprises a mixture of water and alcohol denat.
23. A transparent hair conditioning composition according to any one of statements 19 to 22, comprising a total amount of the or each additional alcohol ranging from about 10 to about 30% by weight of the total composition, preferably from about 10 to about 20%.
24. A transparent hair conditioning composition according to any preceding statement, further comprising one or more glyceryl esters (excluding fats and oils—according to the INCI grouping).
25. A transparent hair conditioning composition according to statement 24, wherein the or each glyceryl ester is selected from Polyglyceryl-3 Caprate/Caprylate/Succinate, Polyglyceryl-6 Caprylate, Polyglyceryl-3 Cocoate, Polyglyceryl-6 Ricinoleate, Polyglyceryl-4 Caprate and mixtures thereof.
26. A transparent hair conditioning composition according to statement 24 or 25, wherein the or each glyceryl ester is a mixture of 2, 3 or 4 of Polyglyceryl-3 Caprate/Caprylate/Succinate, Polyglyceryl-6 Caprylate, Polyglyceryl-3 Cocoate, Polyglyceryl-6 Ricinoleate, Polyglyceryl-4 Caprate.
27. A transparent hair conditioning composition according to statement 24 or 25, wherein the or each glyceryl ester is a mixture of Polyglyceryl-3 Caprate/Caprylate/Succinate, Polyglyceryl-6 Caprylate, Polyglyceryl-3 Cocoate, Polyglyceryl-6 Ricinoleate, Polyglyceryl-4 Caprate.
28. A transparent hair conditioning composition according to any one of statements 24 to 27, comprising a total amount of the or each glyceryl ester ranging from about 0.5 to about 20% by weight of the total composition, preferably from about 1.0 to about 15% by weight of the total composition.
29. A transparent hair conditioning composition according to any preceding statement, further comprising one or more benefit agents.
30. A transparent hair conditioning composition according to statement 29, wherein the or each benefit agent is selected from antidandruff agents, preservatives, UV filters, fats and oils, thickeners, polymers, humectants, vitamins and/or provitamins, hair structuring agents, hair care agents, hair restorers and agents for combating hair loss.
31. A transparent hair conditioning composition according to statement 29 or 30, wherein the benefit agents are selected protein hydrolysates, fats and oils, vitamins, amino acids and/or the derivatives thereof, plant extracts, cationic polymers and hair structuring agents.
32. A transparent hair conditioning composition according to any one of statements 29 to 31, wherein the benefit agent comprises one or more vegetable, animal, mineral and synthetic oils.
33. A transparent hair conditioning composition according to statement 32, wherein the oil is Persea Gratissima (Avocado) Oil.
34. A transparent hair conditioning composition according to statement 32 or 33, comprising a total amount of the or each oil ranging from about 0.01 to about 1.0% by weight of the total composition, preferably from about 0.01 to about 0.5% by weight of the total composition
35. A transparent hair conditioning composition according to any preceding statement, in the form of a spray.
36. A cosmetic preparation comprising the transparent hair conditioning composition of any one of statements 1 to 35.
37. A cosmetic preparation according to statement 36, in a transparent package.
38. A cosmetic preparation according to claim 36 or 37, wherein the preparation packaging is suitable for dispensing the agent in the form of uniform, small droplets.
39. A method of conditioning keratin fibers, comprising contacting a keratin fiber with a conditioning-effective amount of the transparent hair conditioning composition of any preceding statement.
40. A method of conditioning keratin fibers, comprising contacting a plurality of keratin fibers with an amount of the composition as defined in any one of statements 1 to 38, effective to increase the volume of the keratin fibers on styling.
41. A method for conditioning keratin fibers, wherein the hair composition according to statement 35 is spray-applied onto wet or dry keratin fibers and left on the keratin fibers until they are next washed.

### EXAMPLE

The following is an example of the hair conditioning composition according to the present disclosure. The composition is in liquid form and is suitable for application as a spray.

| Ingredient (INCI name) | Quantity - wt % |
|---|---|
| Aqua | ad 100 |
| Alcohol denat. | 10-20 |
| Glycerin | 1.0-5.0 |
| Bis-(Isostearoyl/Oleoyl Isopropyl) Dimonium | 0.5-2.0 |
| Methosulfate | |
| Polyglyceryl 1-3 Caprate/Caprylate/Succinate | 0.5-2.5 |
| Polyglyceryl-6 Caprylate | 0.5-2.5 |
| Polyglyceryl-3 Cocoate | 0.5-2.5 |
| Polyglyceryl-6 Ricinoleate | 0.5-2.5 |
| Polyglyceryl-4 Caprate | 0.5-2.5 |
| Brassicamidopropyl Dimethylamine | 0.5-1.5 |
| Succinic Acid | 0.5-2.5 |
| Parfum | ad |
| Natural Oil eg. Persea Gratissima Oil | 0.01-0.5 |
| Propylene Glycol | 0.01-1.5 |

It will be appreciated that the present disclosure may be modified within the scope of the appended claims.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A transparent hair conditioning composition comprising bis-(isostearoyl/oleoyl isopropyl) dimonium methosulfate, wherein the composition is silicone-free.

2. A transparent hair conditioning composition according to claim 1, comprising bis-(isostearoyl/oleoyl isopropyl) dimonium methosulfate in an amount ranging from about 0.01 to about 5.0% by weight of the total composition.

3. A transparent hair conditioning composition according to claim 1, further comprising at least one dicarboxylic acid, or a salt or derivative thereof, optionally selected from oxalic acid, malonic acid, succinic acid, glutaric acid, adipic acid, pimelic acid, azelaic acid, maleic acid, fumaric acid, sorbic acid and mixtures thereof.

4. A transparent hair conditioning composition according to claim 3, wherein the dicarboxylic acid or salt or derivative thereof is succinic acid.

5. A transparent hair conditioning composition according to claim 2, comprising a total amount of dicarboxylic acid or salt or derivative thereof in an amount ranging from about 0.01 to about 5.0% by weight of the total composition.

6. A transparent hair conditioning composition according to claim 1, further comprising at least one additional cationic surfactant selected from an amidoamine, a permanent cationic amidoamine, and mixtures thereof.

7. A transparent hair conditioning composition according to claim 6, comprising an amidoamine selected from Brassicamidopropyl dimethylamine, lauramidopropyl dimethylamine, myristamidopropyl dimethylamine, stearamidopropyl dimethylamine, cocamidopropyl dimethylamine, ricinolamidopropyl dimethylamine, isostearamidopropyl dimethylamine, oleamidopropyl dimethylamine, behenamidopropyl dimethylamine and palmamidopropyl dimethylamine.

8. A transparent hair conditioning composition according to claim 6, comprising a permanent cationic amidoamine selected from Cetrimonium Methosulfate, Quaternium-33, behenamidopropyl ethyldimonium ethosulfate, behenamidopropyl PG-dimonium chloride, oleamidopropyl ethyldimonium ethosulfate, oleamidopropyl PG-dimonium chloride, cocamidopropyl ethyldimonium ethosulfate, cocamidopropyltrimonium chloride, ricinoleamidopropylethyldimonium ethosulfate, rinoleamidopropyltrimonium chloride, ricinoleamidopropyltrimonium methosulfate, stearamidopropyl ethyldimonium ethosulfate, stearamidopropyl trimonium methosulfate, undecyleneamidopropyltrimonium methosulfate, lauramidopropyl PG-dimonium chloride, and canolamidopropyl ethyldimonium ethosulfate.

9. A transparent hair conditioning composition according to claim 6, comprising a total amount of additional cationic surfactant ranging from about 0.1 to about 2.0% by weight of the total composition.

10. A transparent hair conditioning composition according to claim 1, further comprising an aqueous-alcoholic carrier, wherein the alcoholic component of the aqueous-alcoholic carrier is selected from alcohol, alcohol denat, propyl alcohol, and isopropyl alcohol.

11. A transparent hair conditioning composition according to claim 1, further comprising a glyceryl ester selected from Polyglyceryl-3 Caprate/Caprylate/Succinate, Polyglyceryl-6 Caprylate, Polyglyceryl-3 Cocoate, Polyglyceryl-6 Ricinoleate, and Polyglyceryl-4 Caprate.

12. A transparent hair conditioning composition according to claim 11, comprising a mixture of 2, 3 or 4 of Polyglyceryl-3 Caprate/Caprylate/Succinate, Polyglyceryl-6 Caprylate, Polyglyceryl-3 Cocoate, Polyglyceryl-6 Ricinoleate, Polyglyceryl-4 Caprate or a mixture of Polyglyceryl-3 Caprate/Caprylate/Succinate, Polyglyceryl-6 Caprylate, Polyglyceryl-3 Cocoate, Polyglyceryl-6 Ricinoleate, Polyglyceryl-4 Caprate.

13. A transparent hair conditioning composition according to claim 1, in the form of a spray.

14. A method of conditioning keratin fibers, comprising contacting a keratin fiber with a conditioning-effective amount of the transparent hair conditioning composition of claim 1.

15. A cosmetic preparation comprising a package comprising the transparent hair conditioning composition of claim 1.

16. The cosmetic preparation according to claim 15, comprising a clear package.
